(19) United States
(12) Patent Application Publication
(10) Pub. No.: US 2024/0285872 A1
(43) Pub. Date: Aug. 29, 2024

(54) MULTI-NEEDLE MODULE FOR SKIN TREATMENT

(71) Applicant: NOBAMEDI CO., LTD., Yongin-si, Gyeonggi-do (KR)

(72) Inventor: Hyun Mee NOH, Yongin-si, Gyeonggi-do (KR)

(73) Assignee: NOBAMEDI CO., LTD., Yongin-si, Gyeonggi-do (KR)

(21) Appl. No.: 18/564,983

(22) PCT Filed: Dec. 2, 2021
(86) PCT No.: PCT/KR2021/018089

§ 371 Date: Nov. 28, 2023

(30) Foreign Application Priority Data
Nov. 19, 2021 (KR) 10-2021-0160660

(51) Int. Cl. A61M 5/32; A61M 5/34

(52) U.S. Cl. CPC A61M 5/3298; A61M 5/349

(57) ABSTRACT

The present invention relates to a multi-needle module for skin treatment, including a needle; a cap part including a front plate including, formed therein, a needle hole through which the needle is passed and an injection hole through which adhesive is injected, and a receiving portion including an opening portion formed at a rear side and an empty space formed therein; a housing including an opening portion coupled to the rear opening portion of the cap part, a space formed therein, and a syringe coupling part connected to a syringe; and a connector inserted into the housing, and includes a concave adhesive injection part formed on a side to be filled with adhesive, and mounted with a plurality of needles on an upper portion thereof, in which the connector is spaced apart from the front plate of the cap part so as to form the adhesive injection part.

### TECHNICAL FIELD

The present disclosure relates to a multi-needle module for skin treatment, and more specifically, to a multi-needle module for skin treatment that allows skin treatment to be performed smoothly without loss of drug.

### DESCRIPTION OF RELATED ART

In general, the treatment method (hereinafter referred to as ‘multi-hole treatment method’), which involves forming a number of microscopic holes in the skin and allowing drugs, etc. to be injected into the body, is widely used to eliminate various skin problems such as wrinkles, blemishes, stretch marks, acne, freckles, pigmentation, etc., maintain hair health, improve hair loss, treat obesity, and so on.

This multi-hole treatment method can use the self-regenerative power of damaged skin tissue to grow new skin or allow drugs, etc. to penetrate into the body through tiny holes, thereby achieving rapid results.

The related multi-needle module for skin treatment has the problem of requiring additional parts such as vacuum pumps and suction pipes for suctioning the skin, which not only increases high manufacturing costs, but also makes the procedure rather cumbersome for the operator.

Korean Patent No. 10-1508067 discloses “Multi-needle module for skin treatment.” Referring to the related art, the configuration includes a needle hub including needle through holes formed therein, a syringe guider coupled to the needle hub, and a needle guider inserted into the syringe guider and the needle guider and equipped with a plurality of needles, in which the syringe guider and the needle guider are integrally combined with medical adhesive.

That is, after inserting a needle hub 40 into a jig 70, needle guiders 20 are stacked such that needle through holes 43 and needle coupling holes 23 are aligned inside the needle hub 40, and then needles 30 are inserted into the needle through holes 43 and needle coupling holes 23 aligned with each other such that front ends of the needles 30 are supported in contact with an inner bottom surface of the jig 70, and then the syringe guider 10 is stacked on the needle guiders 20, and then an integrated structure is manufactured as medical adhesive 5 is poured onto an upper part of the syringe guider 10 to be filled up to the needle through holes 43, and then the medical adhesive 5 is hardened. Therefore, bonding is performed two times, and there is a problem in that the manufacturing process is complicated and production efficiency is low due to use of manual manufacturing, because, in order to assemble a needle module, it requires inserting the needle hub, inserting the needles, and performing a primary bonding, and then inserting the needle guiders, inserting the syringe guider, injecting solution for a secondary bonding through the bonding injection holes on the rear side, and then removing the result from the jig.

Further, in the related art, assembling the needle module requires use of the jig, and it is also necessary to inject the medical adhesive from the syringe guider at the rear side to fill the needle through holes, but due to the complex structure, there is a problem that the medical adhesive cannot be injected deep into the interior, resulting in defective products.

### SUMMARY OF INVENTION

### Technical Problem

The present disclosure has been proposed to solve the problems of the related art, and an object of the present disclosure is to provide a multi-needle module for skin treatment, which has a simple structure such that a bond can be sufficiently injected from the front of the module to the rear part of the module, so that the adhesion between each component can be made strong and simple, thereby speeding up the assembly process and improving product reliability.

### Solution to Problem

It is an object of the present disclosure to provide a multi-needle module for skin treatment, which is connected to a syringe and mounted on an injection device, and may be achieved by a multi-needle module for skin treatment including a needle: a cap part including a front plate including, formed therein, a needle hole through which the needle is passed and an injection hole through which adhesive is injected, and a receiving portion including an opening formed at a rear side and an empty space formed therein: a housing including an opening portion coupled to the rear opening portion of the cap part, a space formed therein, and a syringe coupling part connected to a syringe; and a connector inserted into the housing, and includes a concave adhesive injection part formed on a side to be filled with adhesive, and mounted with a plurality of needles on an upper portion thereof, in which the connector may be spaced apart from the front plate of the cap part so as to form the adhesive injection part.

The housing may include an inlet hole penetrating through a center of a bottom and communicating with the syringe coupling part, and a plurality of distribution passages communicating with the inlet hole, in which one end of the needle may be inserted into an end of each distribution passage.

The connector may be in close contact with an inner bottom of the housing and may include an insertion part protruding from a side to be closely fitted in an inner wall.

The connector may be inserted into the receiving portion of the cap part, while being spaced apart from a lower portion of the front plate by a certain distance so as to form a gap to be filled with adhesive, the opening portion of the cap part may be in close contact with the opening portion of the housing, and the needle, the cap part, the housing, and the connector may be all adhesively fixed at once by the adhesive B injected into the gap.

An end of the insertion jaw of the opening portion of the cap part may be formed in a length that reaches 1/10 to 9/10 of an inner length of the opening portion of the housing, and the adhesive may be filled in a concave portion corresponding to a thickness of the insertion jaw of the opening portion of the cap part so as to bond the inner surface of the opening portion of the housing.

The syringe coupling part may include a syringe coupling hole formed at one end, a passage formed inside, and a locking protrusion formed on an outer peripheral surface, and the locking protrusion may be coupled and fixed to the injection device.

The syringe coupling part may include a locking surface having a flat straight surface formed on at least a portion of the outer peripheral surface.

The locking protrusion formed on the outer peripheral surface of the syringe coupling part may include a straight part having a flat straight surface formed on at least a portion of an outer peripheral surface of the locking protrusion.

### Effects of Invention

According to the present disclosure, the configuration can be simplified as there is no need for a jig when assembling the needle module, the injection process is easier as the adhesive is injected through the injection hole formed on the front of the needle module, and the injected adhesive can be used to bond the cap part and the housing on both sides through the connection part, so that each component can be adhered firmly and easily, which has the effect of reducing assembly man-hours.

In addition, there is an effect of increasing product reliability because the syringe coupling part is prevented from spinning.

### BRIEF DESCRIPTION OF THE DRAWINGS

FIG. 1 is a perspective view of a multi-needle module for skin treatment according to the present disclosure.

FIG. 2 is an exploded cross-sectional view of the multi-needle module for skin treatment according to the present disclosure.

FIG. 3 is a cross-sectional view showing a distribution passage 240 of the multi-needle module for skin treatment according to the present disclosure.

FIG. 4 is a cross-sectional view of the multi-needle module for skin treatment in assembled state according to the present disclosure.

FIG. 5A, FIG. 5B, and 5C are views showing a syringe coupling part according to another embodiment.

FIG. 6 is a view showing a state in which the multi-needle module for skin treatment according to the present disclosure is mounted on an injection device for use.

FIG. 7 is a perspective view of a multi-needle module for skin treatment according to another embodiment of the present disclosure.

FIG. 8 is a perspective view of a multi-needle module for skin treatment according to still another embodiment of the present disclosure.

### DETAILED DESCRIPTION FOR EMBODYING INVENTION

Hereinafter, the preferred embodiment will be described in detail below based on the attached drawings.

The examples to be described below are intended to explain the present disclosure in detail such that a person skilled in the art can easily practice the present disclosure, and do not mean that the technical idea and scope of the disclosure are limited.

In addition, it is to be noted that the size or shape of the components shown in the drawings may be exaggerated for clarity and convenience of explanation, and terms specifically defined in consideration of the configuration and operation of the present disclosure may vary according to the intention or custom of the user or operator, and these terms should be defined based on the content throughout this specification.

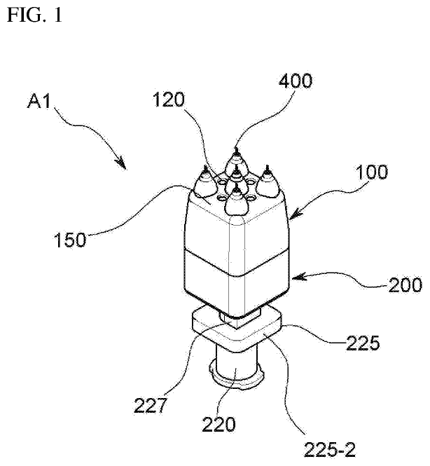

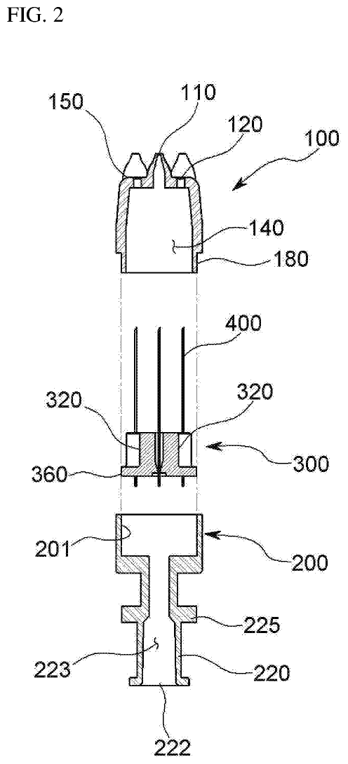

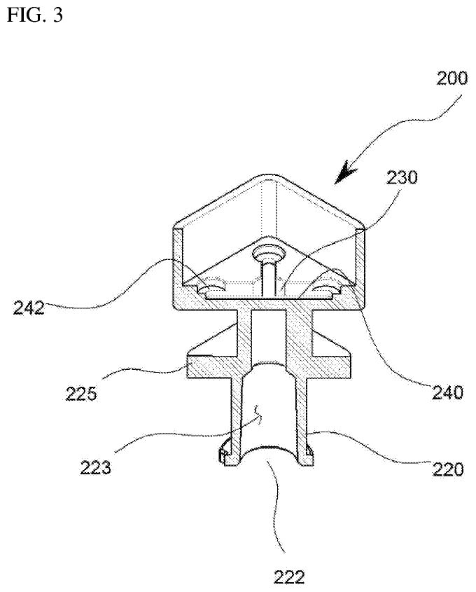

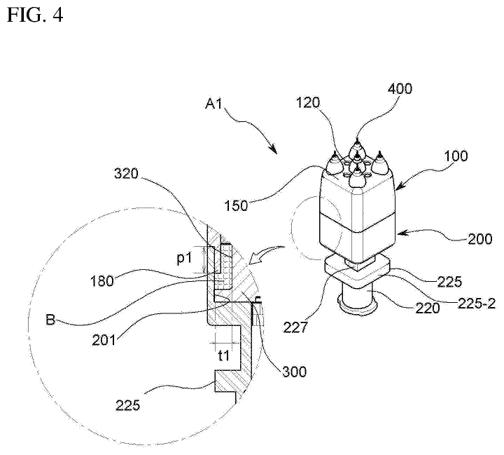

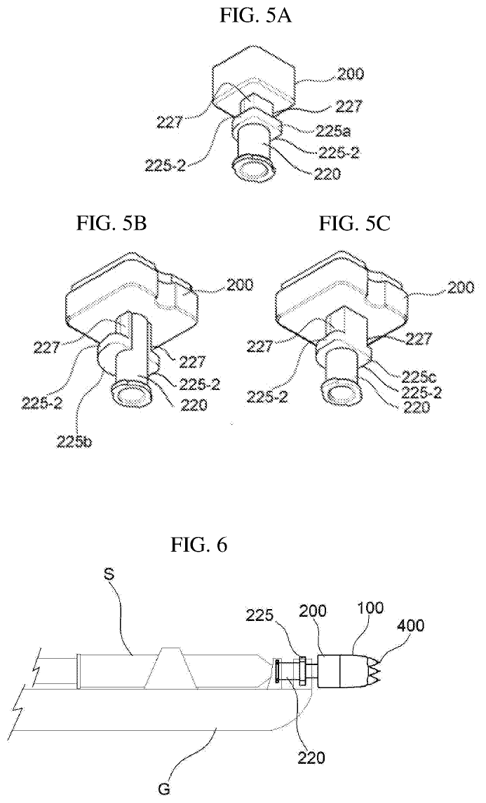

In the accompanying drawings, FIG. 1 is a perspective view of a multi-needle module for skin treatment according to the present disclosure, FIG. 2 is an exploded cross-sectional view of the multi-needle module for skin treatment according to the present disclosure, FIG. 3 is a view showing a syringe coupling part according to another embodiment, FIG. 4 is a view showing a state in which the multi-needle module for skin treatment according to the present disclosure is mounted on an injection device for use, FIG. 5A, FIG. 5B, and FIG. 5C are perspective views of a multi-needle module for skin treatment according to another embodiment of the present disclosure, and FIG. 6 is a perspective view of a multi-needle module for skin treatment according to still another embodiment of the present disclosure.

As shown in FIGS. 1 to 6, a multi-needle module A1 for skin treatment according to the present disclosure is connected to a syringe S and mounted on an injection device, and mainly includes in its configuration a needle 400, a cap part 100, a housing 200, and a connector 300.

There are one or more needles 400.

The cap part 100 includes a front plate 150 including, formed therein, a needle hole 110 through which the needle 400 is passed and an injection hole 120 through which adhesive is injected, and a receiving portion 140 including an opening portion 130 formed at a rear side and an empty space formed therein. The cap part 100 may include a needle guide pipe (not shown) therein, which may extend to the needle hole 110 through which the needle 400 is passed, and the needle 400 may be stably assembled through the needle guide pipe.

The housing 200 includes an opening portion coupled to the rear opening portion of the cap part 100, a space formed therein, and a syringe coupling part 220 connected to the syringe S.

The housing 200 includes an inlet hole 230 penetrated through a center of the bottom and communicating with the syringe coupling part 220, and a plurality of distribution passages 240 communicating with the inlet hole 230, in which a liquid receiving portion 242, into which one end of the needle 400 is inserted, is formed at an end of each distribution passage.

The housing 200 includes an inlet hole 230 penetrated through a center of the bottom and communicating with the syringe coupling part 220, and a plurality of distribution passages 240 communicating with the inlet hole 230, in which a liquid receiving portion 242, into which one end of the needle 400 is inserted, is formed at an end of each distribution passage.

The arrangement of the distribution passages 240 may be provided in various forms according to the number of needles, and in such a form that allows the injected drug to be smoothly distributed and injected through the needles. The arrangement may be provided in various forms according to the number of needles 400 and is not limited to a specific example.

The connector 300 is inserted into the housing 200, and includes an adhesive injection part 320 formed on a side to be filled with adhesive B, and is mounted with a plurality of needles 400 on the upper portion.

The adhesive injection part 320 is inserted into the housing 200 to form a space to be filled with the adhesive. The shape of the adhesive injection part 320 is not limited to a specific shape, and includes any shape that is recessed inward to form a space to be filled with the adhesive.

Preferably, it may be formed to be inclined from top to bottom, in which the upper portion where the adhesive B flows in is wider to facilitate the inflow of the adhesive, and the adhesive B can gravitate along the inclined surface to the bottom, such that the adhesive B can be relatively easily filled in the upper space of the connector 300 formed inside the cap and in a gap.

Referring to FIG. 4, the connector 300 is inserted into the receiving portion 140 of the cap part 100, while having a gap with a lower portion of the front plate 150 by a certain distance, so that the adhesive B is filled in the gap. The adhesive B can be easily filled in the gap and the upper space of the connector 300 inside the cap.

The connector 300 is in close contact with an inner bottom of the housing 200, and includes an insertion part 360 protruding from the side to be closely fitted in an inner wall of the housing 200.

According to an embodiment, the opening portion of the cap part 100 is in close contact with the opening portion of the housing 200, the cap part 100 and the housing 200 may be bonded by the adhesive B injected inside, and the adhesive B may adhesively fix the needle 400 of the connector 300, the cap part 100, the housing 200, and the connector 300 all at once.

According to another embodiment, an outer surface of the opening portion of the cap part 100 is indented to form an insertion jaw 180, and the insertion jaw 180 is tightly coupled with the inner surface of the opening portion 201 of the housing 200.

Preferably, referring to FIG. 4, the opening portion 130 of the cap part 100 is formed in a length such that it reaches the 1/10 to 9/10 position (p1) of an inner length of the opening portion 201 of the housing 200. Therefore, the adhesive B can be filled in a concave area corresponding to a thickness t1 of the opening portion of the cap part 100, so that the inner surface of the opening portion 201 of the housing 200 can be adhered thereto, thereby bonding the cap part 100, the housing 200, and the connector 300 together. In addition, the inner surface of the opening portion of the cap part 100 may be cut outward to form an insertion jaw, in which case the outer surface of the opening portion 201 of the housing 200 may be indented to form an insertion jaw corresponding to the insertion jaw of the cap part 100, so that the insertion jaw of the cap part 100 and the insertion jaw of the housing 200 are tightly coupled with each other. Therefore, even if strong pressure is exerted during the process of filling the gap with the adhesive B, the situation where the adhesive B leaks out of the multi-needle module for skin treatment through the connection part between the cap part 100 and the housing 200 can be prevented.

The housing 200 includes the syringe coupling part 220 communicating with the inlet hole 230 formed through the center of the bottom.

Referring to FIG. 6, the syringe coupling part 220 includes a syringe coupling hole 222 formed at one end, and a passage 223 formed therein.

The syringe coupling part 220 is formed in a circular or oval shape, and includes a locking surface 227 having a flat straight surface formed on at least a portion of an outer peripheral surface to prevent spinning. Preferably, locking surfaces 227 in the shape of flat straight surface are formed on both sides of the syringe coupling part 220. When coupled to a syringe having a shape corresponding to the circular or oval shape of the syringe coupling part 220, the syringe coupling part 220 would spin around after being coupled to the syringe, but due to the presence of the flat straight locking surfaces 227 on both sides of the syringe coupling part 220, the syringe coupling part 220 is prevented from rotating even after being attached to the syringe allowing it to be stably coupled and used.

Meanwhile, the syringe coupling part 220 includes locking protrusions 225, 225a, 225b, and 225c formed on its outer peripheral surface.

The locking protrusions 225, 225a, 225b, and 225c may be formed in various shapes such as square, circular, and oval, and protrude beyond the syringe coupling part 220, and the locking protrusions 225 protruding as described above are coupled to the injection device G.

As shown in FIG. 5A, the locking protrusion 225a may include a straight part 225-2 having a flat straight surface formed on at least a portion of the outer peripheral surface so as to prevent rotating. Preferably, straight parts 225-2 in the shape of flat straight surface are formed on both sides of the locking protrusion 225a. When coupled to a syringe having a shape corresponding to the circular or oval shape of the locking protrusion 225a, the locking protrusion 225a would spin around after being coupled to the syringe, but due to the presence of the straight parts 225-2 in the shape of flat straight surface on both sides of the locking protrusion 225a, the locking protrusion 225a is prevented from spinning around even after being coupled to the syringe allowing it to be stably coupled and used.

As shown in FIG. 5B, the locking surface 227 and the locking protrusion 225b may be formed by thickening the outer peripheral surface of the syringe coupling part 220 except for a certain portion, and the syringe coupling part 220 is formed in a circular or oval shape, and includes the locking surface 227 having a flat straight surface formed on at least a portion of the outer peripheral surface to prevent spinning. The locking protrusion 225b has a circular or oval shape, and includes the straight part 225-2 having a flat straight surface formed on at least a portion of the outer peripheral surface to prevent rotation.

As shown in FIG. 5C, the locking surface 227 and the locking protrusion 225c may be formed by thickening the outer peripheral surface of the syringe coupling part 220, and the locking surface 227 having a flat straight surface formed on at least a portion of the outer peripheral surface prevents spinning. In addition, the locking protrusion 225c has a circular or oval shape, and includes the straight part 225-2 having a flat straight surface formed on at least a portion of the outer peripheral surface to prevent rotation. Therefore, the syringe coupling part 220 and the locking protrusions 225, 225a, 225b, and 225c are fitted into mounting grooves (not shown) formed on the injection device G, so that the multi-needle module of the present disclosure is fixed so as not to be separated and can be stably coupled and prevented from spinning.

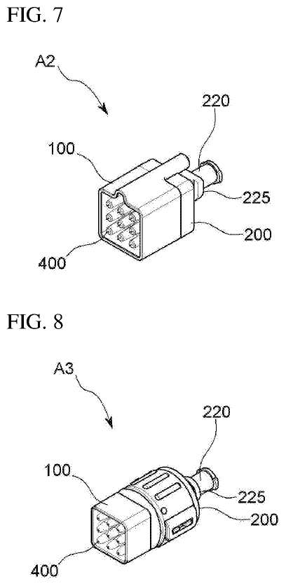

Meanwhile, FIG. 7 is a diagram showing a multi-needle module for skin treatment with 9 needles according to an embodiment.

FIG. 8 is a diagram showing a screw needle type including 9 needles and a housing 200 coupled by a screw coupling method.

In the related art, manufacturing process is complicated because the related needle assembly process involves performing bonding two times, and the needle module is assembled by inserting the needle hub (corresponding to the “connector 300” of the present disclosure), inserting the needles and performing a primary bonding, and inserting the needle guider (corresponding to the “housing 200” of the present disclosure), inserting the syringe guider (corresponding to the “cap part 100” of the present disclosure), and then injecting the secondary bonding liquid through the bonding injection hole on the rear side, and then removing the result from the jig.

In addition, in the related art, because the bonding injection hole is formed at the rear end of the needle and the bond is injected through the bonding injection hole, it is required to modify the shape of the blocking protrusion 225 formed at the rear end of the needle to inject the bond through the bonding injection hole at the rear end of the needle so that the blocking protrusion does not interfere. In addition, in the related art, the needles are manufactured by inserting the needle module in the assembled state into the jig so that the cap part inserted with the needles is positioned in the jig, inserting the connector for fixing the needles and then positioning the needle module with its rear side facing up, injecting the bond through the bond injection hole provided at the rear side of the needle module to fix the needles, and then removing the jig. In order to improve the problem described above, according to the present disclosure, the manufacturing process can be greatly simplified by eliminating the bond injection hole at the rear end of the needle and filling the inside by injecting bond through the front injection hole 120, and also there are no restrictions on the shape of the locking protrusion 225 so that the locking protrusion 225 in various sizes and shapes can be formed.

Although the description has been made in relation to the preferred embodiment, it will be readily apparent to those skilled in the art that various modifications and variations can be made without departing from the gist and scope of the disclosure, and it is obvious that all such changes and modifications fall within the scope of the appended claims.

### INDUSTRIAL APPLICABILITY

The present invention is applicable for use in a multi-needle module for skin treatment without loss of drug when injecting drugs into the skin.

1. A multi-needle module for skin treatment, which is connected to a syringe and mounted on an injection device and comprises:
a needle;
a cap part including a front plate including, formed therein, a needle hole through which the needle is passed and an injection hole through which adhesive is injected, and a receiving portion including an opening formed at a rear side and an empty space formed therein;
a housing including an opening portion coupled to the rear opening portion of the cap part, a space formed therein, and a syringe coupling part connected to a syringe; and
a connector inserted into the housing, and includes a concave adhesive injection part formed on a side to be filled with adhesive, and mounted with a plurality of needles on an upper portion thereof,
wherein the connector is spaced apart from the front plate of the cap part so as to form the adhesive injection part.

2. The multi-needle module for skin treatment according to claim 1, wherein the housing includes an inlet hole penetrating through a center of a bottom and communicating with the syringe coupling part; and
a plurality of distribution passages communicating with the inlet hole, wherein one end of the needle is inserted into an end of each distribution passage.

3. The multi-needle module for skin treatment according to claim 1, wherein the connector is in close contact with an inner bottom of the housing and includes an insertion part protruding from a side to be closely fitted in an inner wall.

4. The multi-needle module for skin treatment according to claim 1, wherein the connector is inserted into the receiving portion of the cap part, while being spaced apart from a lower portion of the front plate by a certain distance so as to form a gap to be filled with adhesive,
the opening portion of the cap part is in close contact with the opening portion of the housing, and
the needle, the cap part, the housing, and the connector are all adhesively fixed at once by the adhesive injected into the gap.

5. The multi-needle module for skin treatment according to claim 1, wherein the opening portion of the cap part is in close contact with the opening portion of the housing,
an outer surface of the opening portion of the cap part is indented to form an insertion jaw, and
the insertion jaw is closely coupled to an inner surface of the opening portion of the housing.

6. The multi-needle module for skin treatment according to claim 5, wherein an end of the insertion jaw of the opening portion of the cap part is formed in a length that reaches 1/10 to 9/10 of an inner length of the opening portion of the housing, and
the adhesive is filled in a concave portion corresponding to a thickness of the insertion jaw of the opening portion of the cap part so as to bond the inner surface of the opening portion of the housing.

7. The multi-needle module for skin treatment according to claim 1, wherein the syringe coupling part includes a syringe coupling hole formed at one end, a passage formed inside, and a locking protrusion formed on an outer peripheral surface, and the locking protrusion can be coupled and fixed to the injection device.

8. The multi-needle module for skin treatment according to claim 7, wherein the syringe coupling part includes a locking surface having a flat straight surface formed on at least a portion of the outer peripheral surface.

9. The multi-needle module for skin treatment according to claim 7, wherein the locking protrusion includes a straight part having a flat straight surface formed on at least a portion of an outer peripheral surface.
